(19) United States
(12) Patent Application Publication
(10) Pub. No.: US 2024/0393238 A1
(43) Pub. Date: Nov. 28, 2024

(54) ULTRA-WIDEBAND TERAHERTZ IMAGING SYSTEM AND IMAGING METHOD

(71) Applicant: Shenzhen Technology University, Shenzhen (CN)

(72) Inventor: Lulu WANG, Shenzhen (CN)

(21) Appl. No.: 18/433,576

(22) Filed: Feb. 6, 2024

(30) Foreign Application Priority Data
May 23, 2023 (CN) 202310585310.3

(51) Int. Cl. G01N 21/3581; A61B 5/0507; G01N 21/3563

(52) U.S. Cl. CPC G01N 21/3581; A61B 5/0507; G01N 21/3563

(57) ABSTRACT

The present invention discloses an ultra-wideband terahertz imaging system and an imaging method. The ultra-wideband terahertz imaging system includes a terahertz wave generator, a terahertz signal emitter, a terahertz biological signal receiver, a terahertz biological signal processor, an image displayer and an intelligent scanning controller. The 3D holographic terahertz imaging system provided by the present invention can perform 2D or 3D image reconstruction on organs, and makes it unnecessary to paste any detector on organs in a manner of non-contact and non-trauma.

### CROSS-REFERENCE TO RELATED PATENT APPLICATION

This application claims priority to and the benefit of Chinese Patent Application No. CN202310585310.3 filed in China on May 23, 2023. The disclosure of the above application is incorporated herein in its entirety by reference.

### FIELD OF THE INVENTION

The present invention relates to the technical field of terahertz imaging, in particular to an ultra-wideband terahertz imaging system and an imaging method.

### BACKGROUND OF THE INVENTION

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Globally, breast cancer is the most frequently diagnosed female cancer and the leading cause of cancer death in females. Early detection and treatment of breast cancer can significantly reduce mortality rate, but its incidence has increased over the past 30 years. Therefore, early detection of breast cancer has become the key to improve the five-year survival rate of breast cancer. Mammography has been considered as the current golden standard medical imaging approach for breast cancer detection. However, the ionizing radiation generated by this method may be harmful to patients and is not suitable for patients with dense breasts and pregnant women. The quality of ultrasound imaging is relatively poor, which poses a challenge in distinguishing early-stage malignant tissue from normal tissue. MRI is the most sensitive method for diagnosing breast cancer, but its cost is high and it cannot accurately locate the mammary gland, which may lead to misdiagnosis. These traditional medical imaging methods have certain limitations, which forces researchers to develop new cancer detection methods.

Compared with conventional breast cancer imaging techniques, terahertz breast imaging has the advantages of non-invasive, non-invasive, non-ionizing radiation, high contrast and survey simplicity, which is expected to become a safe and effective means of routine or auxiliary detection of cancer, and has attracted extensive attention from scholars around the world. However, terahertz imaging technology currently faces some limitations, including long scanning time and limited image resolution, leading to slow progress in its promotion.

### SUMMARY OF THE INVENTION

In order to solve the technical limitation of the terahertz imaging technology in the above-mentioned prior art, the present invention provides an ultra-wideband terahertz imaging system and an imaging method.

The technical scheme adopted in the present invention is as follows:

The present invention provides an ultra-wideband terahertz imaging system comprising:

a terahertz wave generator used to generate a continuous ultra-wideband terahertz signal;
a terahertz signal emitter emitting the continuous ultra-wideband terahertz signal generated by the terahertz wave generator to an organ;
a terahertz biological signal receiver, and used to receive an electromagnetic field around the organ to obtain a scattered electric field;
a terahertz biological signal processor performing multi-dimensional image reconstruction on the received scattered field electric field to obtain a multi-dimensional image of the organ;
an image displayer used to display the multi-dimensional image of the organ; and
an intelligent scanning controller connected with and controlling the terahertz wave generator, the terahertz signal emitter, the terahertz biological signal receiver, the image displayer, and the terahertz biological signal processor.

Specifically, the terahertz signal emitter includes at least one emitting antennas, and the terahertz biological signal receiver includes at least one receiving antennas, and the receiving antenna can adjust the vertical height.

Specifically, the intelligent scanning controller controls the receiving antenna of the terahertz biological signal receiver to move around the organ, so as to perform rotational scanning, or controls the receiving antenna of the terahertz biological signal receiver to move upwards and downwards, so as to perform scanning in a vertical direction, or controls the receiving antenna of the terahertz biological signal receiver to move horizontally, so as to perform scanning in a horizontal direction.

The present invention further provides an imaging method of the ultra-wideband terahertz imaging system, comprising the steps of
S1, controlling the terahertz wave generator to generate a continuous ultra-wideband terahertz signal;
S2, controlling the terahertz signal emitter to uninterruptedly emit an ultra-wideband terahertz signal to the organ;
S3, controlling the terahertz biological signal receiver to receive an electromagnetic field from the organ, so as to obtain a scattered electric field;
S4, controlling the terahertz biological signal processor to perform multi-dimensional image reconstruction on the scattered electric field detected by the terahertz biological signal receiver, so as to obtain a multi-dimensional image of the organ; and
S5, transmitting the multidimensional image of the organ to the image displayer for imaging the tested organ.

Further, S2 includes the sub-steps of
S21, establishing a rectangular coordinate system at the area to be imaged where the organ is situated; and
S22, enabling N_{T} emitting antennas of the terahertz signal emitter that surround the organ or are situated at one or both sides of the organ, to uninterruptedly apply an ultra-wideband terahertz wave signal to the organ, and setting N_{T} as ≥1.

Specifically, when the number of the emitting antennas, N_{T}>1 occurs, and the emitting antennas are distributed in a uniform circular shape, each transmitting antenna makes an excitation to generate an incident electric field in proper order, and an entire incident electric field is a sum of the incident electric fields excited by N emitting antennas.

In the first embodiment, during processing a 2D image, S3 includes the sub-steps of
S31, controlling one receiving antenna of the terahertz biological signal receiver to detect a scattered electric field of at least three receiving positions at the same vertical height of the receiving antenna away from the organ; or controlling at least three receiving antennas at the same vertical height at a distance from the organ to detect a scattered electric field of other receiving positions;
S32, moving the organ out of the detected area to obtain an incident electric field of the emitting antenna; and
S33, based on the incident electric field of the emitting antenna and the scattered electric field detected by the receiving antenna, calculating out a scattered electric field echo.

In the second embodiment, during processing a 3D image, S3 includes the sub-steps of
S31, controlling one receiving antenna of the terahertz biological signal receiver to detect a scattered electric field of at least three receiving positions at the same vertical height of the receiving antenna away from the organ; or controlling at least three receiving antennas at the same vertical height at a distance from the organ to detect a scattered electric field of other receiving positions;
S32, moving the organ out of the detected area to obtain an incident electric field of the emitting antenna;
S33, based on the incident electric field of the emitting antenna and the scattered electric field detected by the receiving antenna, calculating out a scattered electric field echo; and
S34, adjusting a vertical height of the receiving antenna, then re-executing the sub-steps S31 to S33.

In the first embodiment, S4 includes the sub-steps of
S41, establishing a nonlinear mathematical model involving electromagnetic properties such as dielectric constants, conductivity and magnetic permeability of the organ and the scattered electric field echo, based on the distribution of the receiving positions of the receiving antennas, establishing a mathematical model representing an internal structure of the organ;
S42, sequentially comparing the scattered electric field echoes obtained at any two receiving positions among all receiving positions of the receiving antennas situated at the same vertical height, and obtaining information reflecting an amplitude and phase position of the electromagnetic property distribution of the organ; and
S43, according to the continuously-detected electromagnetic property distribution information, extracting a corresponding varied value and curve from the established nonlinear mathematical model and the mathematical model representing the internal structure of the organ, and based on the varied values, reconstructing a 2D image of the organ.

In the second embodiment, S4 includes the sub-steps of
S41, based on the incident electric field of the emitting antenna and the scattered electric field detected by the receiving antenna, calculating out a scattered electric field echo, establishing a nonlinear mathematical model involving electromagnetic properties such as dielectric constants, conductivity and magnetic permeability of the organ and the scattered electric field echoes, and based on the distribution of the receiving positions of the receiving antennas, establishing a mathematical model representing an internal structure of the organ;
S42, sequentially comparing the scattered electric field echoes obtained at any two receiving positions among all receiving positions of the receiving antennas situated at the same vertical height, and obtaining information reflecting an amplitude and phase position of the electromagnetic property distribution of the organ;
S43, sequentially comparing the scattered electric field echoes obtained at any two receiving positions among all receiving positions of the receiving antennas adjusted in height, and situated at the same vertical height, and obtaining information reflecting an amplitude and phase position of the electromagnetic property distribution of the organ; and
S44, according to the continuously-detected electromagnetic property distribution information, extracting a corresponding varied value and curve from the established nonlinear mathematical model and the mathematical model representing the internal structure of the organ, and based on the varied values, reconstructing a 3D image of the organ.

Compared with the prior art, the 3D holographic terahertz imaging system provided by the present invention can perform 2D or 3D image reconstruction on organs, and makes it unnecessary to paste any detector on organs in a manner of non-contact and non-trauma. Based on the imaging system and imaging method of the present invention, it is possible to develop a corresponding medical instrument, which can display a corresponding curve, an image and a value, and is capable of continuous monitoring on a variety of diseases such as breast cancer, skin cancer, etc.

### BRIEF DESCRIPTION OF THE DRAWINGS

FIG. 1 is a block diagram of the imaging system in the example of the present invention.

FIG. 2 is a system conceptual diagram of the imaging system in the example of the present invention.

FIG. 3 is a schematic diagram showing the positions of the emitting antenna and receiving antenna of the imaging system in the example of the present invention.

FIG. 4 is a schematic diagram showing the geometric arrangement of a pair of terahertz biological signal receivers of the imaging system in the example of the present invention.

FIG. 5 is a schematic diagram showing adjustments to the height of the receiving antenna of the imaging system in the example of the present invention.

FIG. 6 is a 2D image (real part) of the 3D breast model in the example of the present invention.

FIG. 7 is a 2D image (imaginary part) of the 3D breast model in the example of the present invention.

FIG. 8 is a 2D reconstructed image (real part) of the 3D breast model in the example of the present invention.

FIG. 9 is a 2D reconstructed image (imaginary part) of the 3D breast model in the example of the present invention.

### DETAILED DESCRIPTION OF SOME EMBODIMENTS

In order to clarify the technical problems, solutions, and beneficial effects to be solved by the present invention, further detailed descriptions of the present invention are provided in conjunction with the accompanying drawings and examples. It should be understood that the specific examples described herein are only intended to explain the present invention and are not intended to pose a limitation on the present invention.

The electromagnetic properties of organs are closely related to a health condition of a human physiological system, and when a disease occur, the dielectric properties (often expressed as a dielectric constant) and electrical conductivity of biological tissues change significantly. There are obvious differences in dielectricity and conductivity between different types of biological tissues, and these differences provide a feasible physical basis for terahertz imaging to detect the physiological and pathological states of organs. Terahertz imaging for mammary glands performs image reconstruction on the internal tissue structure of organs by way of detecting the distribution of scattered electric fields within and around organs under the action of terahertz electric fields, and obtains important characteristics such as dielectric constant distribution and conductivity distribution of organs. Over the past two decades, researchers have conducted extensive researches on terahertz imaging technology for in vitro biological imaging and tumor detection, such as breast imaging and breast tumor detection.

The present invention provides an ultra-wideband terahertz imaging system and an imaging method, and the imaging system makes it unnecessary to paste any detector on organs in a manner of non-contact and non-trauma. Based on the imaging system and imaging method of the present invention, it is possible to develop a corresponding medical instrument, which can display a corresponding curve, image, numerical value, and is capable of continuous monitoring on a variety of diseases such as breast cancer, skin cancer, etc.

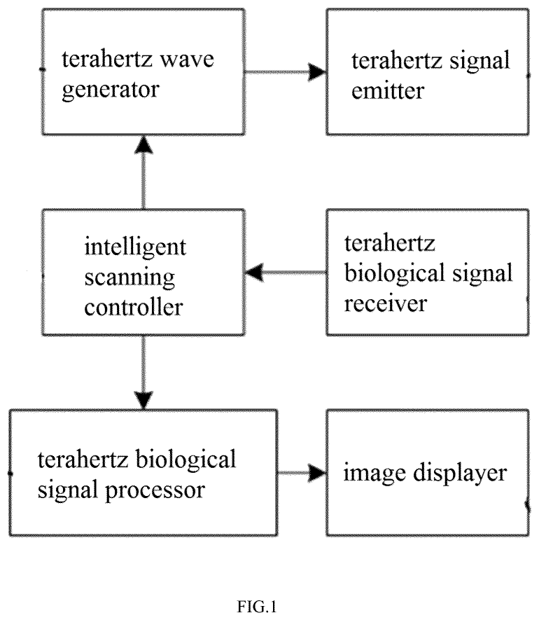

As shown in FIG. 1, the ultra-wideband terahertz imaging system according to the present invention includes a terahertz wave generator, a terahertz signal emitter, a terahertz biological signal receiver, a terahertz biological signal processor, an image displayer and an intelligent scanning controller. The terahertz wave generator is controlled by the intelligent scanning controller and is used to generate a continuous ultra-wideband terahertz signal, which is uninterruptedly applied to the terahertz signal emitter, and the ultra-wideband terahertz signal that is continuously emitted to an organ through the emitting antenna of the terahertz signal emitter will generate an electromagnetic wave field around the organ, and the electromagnetic wave field will cause the organ to generate an electromagnetic field, which will bring an induced magnetic field and/or a scattered magnetic field. The receiving antenna of the terahertz biological signal receiver can detect the scattered magnetic field from the organ, and the detected scattered magnetic field is transmitted to the terahertz biological signal processor, which can perform 2D image reconstruction on the scattered magnetic fields detected by the receiving antenna at different receiving positions at the same vertical height to obtain a 2D image of the organ, and can also perform 3D image reconstruction on the scattered magnetic fields detected by the receiving antenna at different receiving positions at multiple vertical heights to obtain a 3D image of the organ. The image displayer is used to display the reconstructed organ to obtain a multi-dimensional image. The intelligent scanning controller is connected with and controls the terahertz wave generator, the terahertz signal emitter, the terahertz biological signal receiver, the image displayer, and the terahertz biological signal processor. In one example, the terahertz wave generator is a vector network analyzer, which can generate a terahertz wave signal having a frequency range of 0.1 THz-0.17 THz.

As an embodiment, the operating frequency of the imaging system provided by the present invention is ultra-wideband, and its optimal operating frequency range is 0.15 THz.

Specifically, the terahertz signal emitter includes at least one emitting antennas, and the terahertz biological signal receiver includes at least one receiving antennas. The emitting antenna and the receiving antenna are both a terahertz antenna, and the emitting antenna and the receiving antenna may be the same kind of terahertz antennas or different kinds of terahertz antennas. If both are the same kind of antennas, the emitting antenna transmits terahertz wave information, and the receiving antenna receives changes of electromagnetic fields within and around the organ, as well as the distribution state of dielectricity and conductivity.

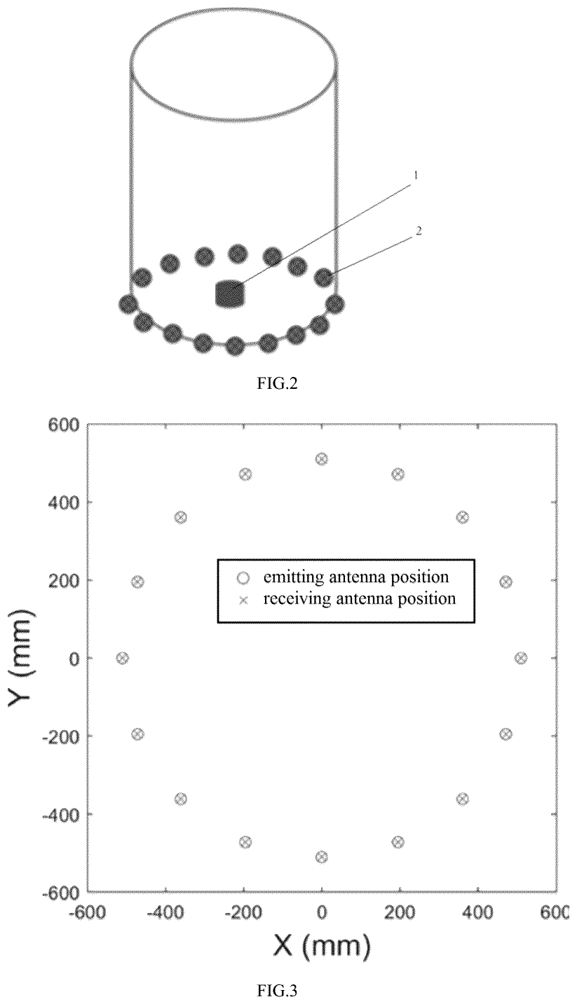

In the example shown in FIG. 2, a plurality of emitting antennas of the terahertz signal emitter are evenly arranged in a circular shape, so that the organ 1 can be surrounded with the circularly-arranged emitting antennas, and the heights of each emitting antenna away from the organ are the same with each other (that is, each emitting antenna lies on the same plane and at the same height).

The receiving antenna 2 of the terahertz biological signal receiver also uses a terahertz antenna, and the number of terahertz antennas acting as a receiving antenna is NR, which is a natural number and ≥1. The terahertz antennas acting as a receiving antenna are evenly arranged around the organ 1, that is, they are evenly arranged in a circular shape, and the distances or heights of each receiving antenna away from the organ 1 are the same with each other, meanwhile the position of the receiving antenna can be adjusted.

In one example, it is possible to arrange only one terahertz antenna, which rotates evenly around the organ by 22.5°, that is, the terahertz antenna can collect 16 messages by way of rotating a circle around the organ. Each terahertz antenna not only acts as an emitting antenna to emit terahertz waves to the organ, but also acts as a receiving antenna to detect the changes of electric fields and the conductivity distribution within and around the organ.

Furthermore, in order to reduce the costs of the imaging system and improve its applicability, it is unnecessary to use an additional medium between the organ and the terahertz antennas and between the terahertz antennas, that is, theses spaces are filled with air.

The operation principle of the holographic terahertz breast imaging system provided by the present invention is as follows. Ultra-wideband terahertz waves are uninterruptedly applied to a certain terahertz emitting antenna, and the ultra-wideband terahertz wave signals reach all receiving antennas by way of transmitting through space. The transmission of the electric field is influenced by the complex conductivity and complex permittivity of the space through which it passes, and it is possible to obtain the information of space complex conductivity or complex permittivity or magnetic permeability by way of comparing respectively in pairs the amplitudes and phase differences of the scattered electric fields of divers receiving antennas. It is achievable to obtain a set of intact measurement data by way of changing the emitting antenna sequentially and utilizing respectively the remaining terahertz antennas for detection. These measurement data are transmitted to the terahertz biological signal processor, which performs image reconstruction on the detected measurement data, so as to obtain an image showing the distribution of conductivity or its ranging within a 2D or 3D interruption by way of reconstruction. During the measurement, the terahertz antenna needs to be rotated evenly to change a detection position.

As an embodiment, the terahertz antenna may be a waveguide antenna, a terahertz patch antenna, a horn antenna, a hypersurface terahertz patch antenna and the likes, and the size of the terahertz antenna is limited by operating frequency, operation environment and materials.

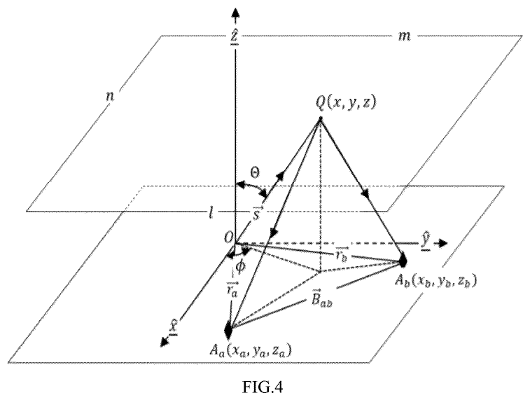

As an embodiment, the positional relations between the terahertz emitting antenna acting as an emitting antenna and the terahertz receiving antenna acting as a receiving antenna includes the following states. The terahertz emitting antenna acting as an emitting antenna and the terahertz receiving antenna acting as a receiving antenna are located together on the same side or respectively on both sides of the organ, at the same height or at different heights; or the terahertz emitting antenna acting as an emitting antenna and the terahertz receiving antenna acting as a receiving antenna are parallel to each other; or the terahertz emitting antenna acting as an emitting antenna coincides with the terahertz receiving antenna acting as a receiving antenna (as shown in FIG. 3); or the terahertz emitting antenna acting as an emitting antenna and the terahertz receiving antenna acting as a receiving antenna are both arranged at a certain angle with the organ.

The terahertz imaging method according to the present invention includes the steps of:
S1, controlling the terahertz wave generator to generate a continuous ultra-wideband terahertz signal;
S2, controlling the terahertz signal emitter to uninterruptedly emit an ultra-wideband terahertz signal to an organ;
S3, controlling the terahertz biological signal receiver to detect an electromagnetic field from the organ, so as to obtain an electromagnetic field signal;
S4, controlling the terahertz biological signal processor to perform multi-dimensional image reconstruction on the electromagnetic field signals received by the terahertz biological signal receiver, so as to obtain a multi-dimensional image of the organ; and
S5, transmitting the multidimensional image of the organ to the image displayer for imaging the tested organ.

In the above step S2, the intelligent scanning controller controls at least one emitting antennas in the terahertz signal to uninterruptedly apply an ultra-wideband terahertz wave to the organ (such as a breast), and the ultra-wideband terahertz wave generates a scattered electric field around the organ (such as a breast), that is, an excitation electric field under an electromagnetic field generates the scattered electric field when it passes through the organ.

The above step S2 includes the sub-steps of:
S21, establishing a rectangular coordinate system at the area to be imaged where the organ is situated, so as to determine a distance between the organ and the emitting antenna as well as the receiving antenna, a position coordinate of the emitting antenna, a position coordinate of the receiving antenna and a number of image points N;
S22, enabling at least one emitting antennas of the terahertz signal emitter to uninterruptedly apply an ultra-wideband terahertz wave signal to the organ, so as to make the signal generate an excitation electric field possibly regarded as a time harmonic electric field within and around the organ, and enabling the excitation electric field to generate an incident electric field under the action of an electromagnetic field when it passes through the organ;

$${\vec{E}}_{inc} ( {\vec{T}}_{xm} ) = ( - \frac{{jk}_{0}}{2 {π}^{2}} ) {\vec{E}}_{0} ( \frac{{e}^{- {jk}_{0} {\vec{R}}_{{\vec{T}}_{xm}}}}{{\vec{R}}_{{\vec{T}}_{xm}}} ) {A}_{N} {B}_{B} h ( θ , ∅ ) \vec{P} ( θ , ∅ )$$

\vec{R}_{\vec{T}}_{ₓₘ} represents a distance vector from an organ model to the emitting antenna situated at \vec{T}ₓₘ, \vec{E}₀ represents an amplitude of a TE10 mode, A_{N} and B_{B} are a length and a width of the antenna, respectively, h(θ, Ø) represents a radiation pattern, and \vec{P}(θ, Ø) represents vector polarization.

As an embodiment, in the above step S22: N_{T} emitting antennas surrounding the organ or located on one or both sides of the organ sequentially emit terahertz wave signals having a specific frequency band.

Specifically, the method of obtaining the scattered electric field echo \vec{E}_{scat} is as follows.

One or more receiving antennas detect the scattered electric field \vec{E}(rᵣ, rᵢ, ω), where rᵢ is a position coordinate of the emitting antenna, rᵣ is a position coordinate of the receiving antenna; when N_{T}>1 occurs, and the emitting antennas are distributed in a uniform circular shape, each transmitting antenna makes an excitation to generate an incident electric field in proper order, and an entire incident electric field is a sum of the incident electric fields excited by N_{T} emitting antennas.

The organ is moved out of the detected area, and the same incident electric field \vec{E}_{inc}(rᵣ, rᵢ, ω) is detected under the premise that the emission source remains unchanged, that is, it is still detected through the same receiving antenna (or the same detection position).

A scattered electric field echo of the organ can be obtained by way of subtracting one of the measurement data derived from the above two steps from another, that is,

$${\vec{E}}_{scat} ( {r}_{r} , {r}_{i} , ω ) = \vec{E} ( {r}_{r} , {r}_{i} , ω ) - {\vec{E}}_{inc} ( {r}_{r} , {r}_{i} , ω )$$

During processing a 2D image, the above step S3 includes the sub-steps of
S31, obtaining a scattered electric field of at least three receiving positions at the same vertical height of the receiving antenna of the terahertz signal emitter at a distance from the organ, that is, at least three receiving antennas may perform detection at three receiving positions at the same vertical height, or one receiving antenna may move to three different receiving positions at the same vertical height for detection;
S32, moving the organ out of the detected area to obtain an incident electric field of the emitting antenna; and
S33, based on the incident electric field of the emitting antenna and the scattered electric field detected by the receiving antenna, calculating out a scattered electric field echo.

If it is necessary to obtain a 3D image, the above sub-steps proceed with the following sub-step of:
S34, adjusting a vertical height of the receiving antenna (specifically relative to a horizontal datum plane where the organ is situated), obtaining a scattered electric field of at least three receiving positions at this vertical of the receiving antenna away from the organ, then re-executing the sub-steps S31 to S33, so as to obtain a scattered electric field echo of another height.

During obtaining a 2D image, the above step S4 includes the sub-steps of:
S41, based on the incident electric field of the emitting antenna and the scattered electric field detected by the receiving antenna, calculating out a scattered electric field echo, establishing a nonlinear mathematical model involving electromagnetic properties such as dielectric constants, conductivity and magnetic permeability of the organ and the scattered electric field echo, based on the distribution of the receiving positions of the receiving antenna, establishing a mathematical model representing an internal structure of the organ;
S42, sequentially comparing the scattered electric field echoes obtained at any two receiving positions among all receiving positions of the receiving antennas situated at the same vertical height, and obtaining information reflecting an amplitude and phase position of the electromagnetic property distribution of the organ; and
S43, according to the continuously-detected electromagnetic property distribution information, extracting a corresponding varied value and curve from the established nonlinear mathematical model and the mathematical model representing the internal structure of the organ, and based on the varied values, reconstructing a 2D image of the organ.

If it is necessary to obtain a 3D image, the above step S4 includes the sub-steps of:
S41, based on an incident electric field of the emitting antenna and a scattered electric field detected by the receiving antenna, calculating out a scattered electric field echo, establishing a nonlinear mathematical model involving electromagnetic properties such as dielectric constants, conductivity and magnetic permeability of the organ and the scattered electric field echoes, and based on the distribution of the receiving positions of the receiving antenna, establishing a mathematical model representing an internal structure of the organ;
S42, sequentially comparing the scattered electric field echoes obtained at any two receiving positions among all receiving positions of the receiving antennas situated at the same vertical height, and obtaining information reflecting an amplitude and phase position of the electromagnetic property distribution of the organ;
S43, sequentially comparing the scattered electric field echoes obtained at any two receiving positions among all receiving positions of the receiving antennas adjusted in height, and situated at the same vertical height, and obtaining information reflecting an amplitude and phase position of the electromagnetic property distribution of the organ; and S44, according to the continuously-detected electromagnetic property distribution
information, extracting a corresponding varied value and curve from the established nonlinear mathematical model and the mathematical model representing the internal structure of the organ, and based on the varied values, reconstructing a 3D image of the organ.

In the above two examples, the step S41 specifically includes the sub-steps of:
establishing a nonlinear mathematical model involving electromagnetic properties such as dielectric constants, conductivity and magnetic permeability of the organ (for example, a breast) and the scattered electric field echoes, and based on the distribution of the receiving positions of the receiving antenna, establishing a mathematical model representing an internal structure of the organ; and
under the action of a mechanism of a terahertz wave penetrating a surface of an organ to induce multiple scattering between different tissues in the organ, obtaining an internal field effect model representing the nonlinear mathematical model and an external field effect model representing the organ.

The internal field effect model is described as follows:

$$\vec{{E}_{T}} ( \vec{{r}^{′}} ) = \vec{{E}_{inc}} ( \vec{{r}^{′}} ) - \frac{j}{4 π ω {μ}_{0}} {\int }_{V} [ ( \vec{{J}_{m}} \cdot ∇ ) ∇ + {k}_{0}^{2} \vec{{J}_{m}} + j ω {μ}_{0} \vec{{J}_{s}} \times ∇ ] G ( \vec{r} , \vec{{r}^{′}} ) dV \qquad ( 2 )$$

In Eq. (2), \vec{E_{inc}} represents an incident electric field, G represents a Green's function, \vec{r′} represents a position vector from a field source point to the scattered electric field, \vec{r′} represents a position vector from a field source point to any point within the organ, k₀ represents a wavenumber in free space, \vec{Jₘ} represents a magnetic current density, \vec{Jₘ}=jωμ₀(μᵣ−1)\vec{E_{T}}, j represents an imaginary part of a complex number, j=\sqrt{−1}, μᵣ represents magnetic conductivity of an organ, μ₀ presents magnetic conductivity of free space, \vec{E_{T}} presents an entire electric field, \vec{E_{T}}=incident electric field+scattered electric field, V presents a volume of a target object.

Its entire electric field mathematical model is as follows:

$${\vec{E}}_{T} ( {\vec{r}}_{m} ) = {\vec{E}}_{inc} ( {\vec{r}}_{m} ) + [ {ε}_{b} {μ}_{b} {ω}^{2} + ∇ ∇ ] {\int }_{V} \vert \frac{\tilde{ε} ( \vec{s} ) - {\tilde{ε}}_{b}}{{\tilde{ε}}_{b}} \vert {\vec{E}}_{T} ( \vec{s} ) G ( {\vec{r}}_{m} , \vec{s} ) dV \qquad ( 3 )$$

In Eq. (3), \vec{E_{inc}} presents an incident electric field, \vec{r}ₘ presents a vector distance from a target point to the terahertz antenna located at \vec{r}ₘ, ∇ represents a divergence operator, G(\vec{r}ₘ,\vec{s}) represents a Green's function,

$$G ( {\vec{r}}_{m} , \vec{s} ) = \frac{{e}^{- j \sqrt{{ε}_{b} {μ}_{b}} ω \vert {\vec{r}}_{m} - \vec{s} \vert}}{4 π \vert {\vec{r}}_{m} - \vec{s} \vert} ,$$

\vec{s} is a position vector from a field source point to any point within an organ.

The external scattered electric field model (external electric field effect model) is as follows:

$${\vec{E}}_{scat} ( {\vec{r}}_{m} ) = \frac{{ε}_{b} {μ}_{b} {ω}^{2}}{4 π} {\int }_{v} \vert \frac{\tilde{ε} ( \vec{s} ) - {\tilde{ε}}_{b}}{{\tilde{ε}}_{b}} \vert [ a {\vec{E}}_{T} ( \vec{s} ) + ( b {\vec{E}}_{T} ( \vec{s} ) \cdot \underline{\hat{{r}_{m}}} ) \underline{\hat{{r}_{m}}} ] G ( {\vec{r}}_{m} ) dV \qquad ( 4 )$$

In Eq. (4), \vec{E}_{scat} represents a scattered electric field (a scattered electric field echo), \vec{r}ₘ represents a vector distance from any target point within an organ to the terahertz antenna located in \vec{r}ₘ, \vec{s} represents a position vector,

$$a = 1 - \frac{j}{\sqrt{{ε}_{b} {μ}_{b}} {ω}_{R}} - \frac{1}{{ε}_{b} {μ}_{b} {ω}^{2} {R}^{2}} , b = - 1 + \frac{3 j}{\sqrt{{ε}_{b} {μ}_{b}} ω R} + \frac{3}{{ε}_{b} {μ}_{b} {ω}^{2} {R}^{2}} ,$$

R represents a distance between a scattering source and a target point.

Further, if providing a≈1, b≈−1, the scattered electric field model may be described as follows:

$${\vec{E}}_{scat} ( {\vec{r}}_{m} ) ≅ \frac{{ε}_{b} {μ}_{b} {ω}^{2}}{4 π} {\int }_{V} \vert \frac{\tilde{ε} ( \vec{s} ) - {\tilde{ε}}_{b}}{{\tilde{ε}}_{b}} \vert [ {\vec{E}}_{T} + ( {\vec{E}}_{T} \cdot \underline{\hat{{r}_{m}}} ) \underline{\hat{{r}_{m}}} ] G ( {\vec{r}}_{m} , \vec{s} ) dV \qquad ( 5 )$$

The nonlinear mathematical model is obtained in combination with the internal electric field effect model and the external electric field effect model.

$${\vec{E}}_{scat} ( {\vec{r}}_{m} ) = \frac{{ε}_{b} {μ}_{b} {ω}^{2}}{4 π} {\int }_{V} \vert \frac{\tilde{ε} ( \vec{s} ) - {\tilde{ε}}_{b}}{{\tilde{ε}}_{b}} \vert {\vec{E}}_{T} ( \vec{s} ) G ( {\vec{r}}_{m} , \vec{s} ) dV \qquad ( 6 )$$

S42 specifically includes the following sub-steps.

Calculating a visible function of any two receiving antenna positions (\vec{rₐ},\vec{r_{b}}) within all receiving antenna positions at the same vertical height (that is, equal vertical distances from the positions to the organ in the same plane); calculating a visible function of any two receiving positions (\vec{rₐ},\vec{r_{b}}) within all receiving antenna positions.

$$\vec{V} ( \vec{{r}_{a}} , \vec{{r}_{b}} ) = 〈 {\vec{E}}_{scat} ( \vec{{r}_{a}} ) \cdot {\vec{E}}_{scat}^{*} ( \vec{{r}_{b}} ) 〉 \qquad ( 7 )$$

In Eq. (7), * presents a complex conjugation, < > presents average time, the visible function \vec{V} contains a phase position delay and/or an amplitude difference detected at any two receiving antenna positions (\vec{rₐ},\vec{r_{b}}).

Sequentially calculating the visible functions of the received signals at any two receiving antenna positions, so as to obtain an entire visible function detected at all receiving positions; when the receiving antenna has N_{R} receiving positions, and N_{R} is a natural number set as N_{R}≥3, thus the entire visible function is a sum of the visible functions detected at N_{R}(N_{R}−1) receiving antenna locations.

Obtaining a 2D image of the organ by way of performing an inverse Fourier transform on the entire visible function detected at all receiving antenna locations.

We shall explain the specific process of the 2D image reconstruction of the organ in combination with the following figures.

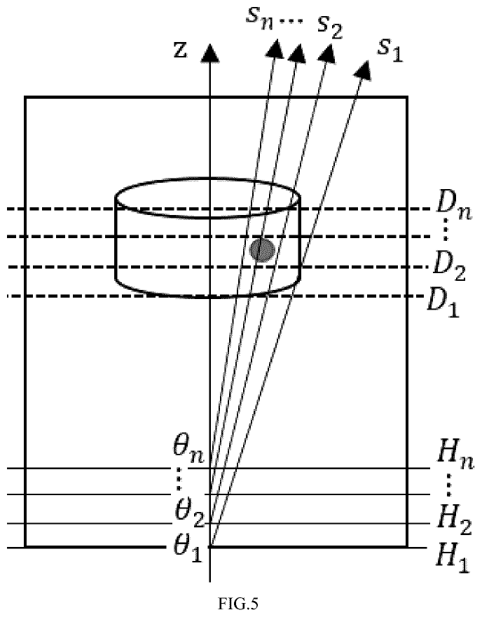

As shown in FIG. 4, provided that a certain point Q(x, y, z) is situated within the organ, by Eq. (7) representing the visible function of the scattered electric field arranging from this point Q(x, y, z) to the receiving position of any two receiving antennas located at \vec{rₐ} and \vec{r_{b}} by Eq. (7), that is, establishing a mathematical model for representation.

The scattering intensity of the organ (breast) at the \vec{s} position is represented as follows.

$$I ( \vec{s} ) = {( \frac{{k}_{0}^{2}}{4 π} )}^{2} {\vert {ε}_{r} ( \vec{s} ) - {ε}_{0} \vert}^{2} {\vec{E}}_{T} ( \vec{s} ) \cdot {\vec{E}}_{T}^{*} ( \vec{s} ) \qquad ( 8 )$$

The volume integral of the visible function of the organ (breast) is as follows.

$$\vec{V} ( \vec{{r}_{a}} , \vec{{r}_{b}} ) = \qquad ( 9 )$$

$${( \frac{{ε}_{b} {μ}_{b} {ω}^{2}}{4 π} )}^{2} {\int \int \int }_{V} {\vert \frac{\tilde{ε} ( \vec{s} ) - {\tilde{ε}}_{b}}{{\tilde{ε}}_{b}} \vert}^{2} {\vec{E}}_{T} ( \vec{s} ) \cdot {\vec{E}}_{T}^{*} ( \vec{s} ) \frac{{e}^{- j \sqrt{{ε}_{b} {μ}_{b}} ω \vert \vec{{r}_{a}} - \vec{{r}_{b}} \vert \cdot \underline{\hat{s}}}}{{s}^{2}} dV$$

Eq. (9) is substituted into Eq. (7) to attain the following equation.

$$\vec{V} ( \vec{{D}_{ab}} ) = {\int \int \int }_{V} I ( \vec{s} ) \frac{{e}^{- j 2 π \vec{{D}_{ab}} \cdot \underline{\hat{s}}}}{{s}^{2}} dV \qquad ( 10 )$$

In Eq. (10), \vec{D_{ab}}=(\vec{rₐ}−\vec{r_{b}})/λ_{b}, λ_{b} is an operating wavelength, ŝ is a unit vector in a spherical coordinate system, ŝ=sin θ cos ϕ\hat{x}+sin θ sin ϕŷ+cos θ\hat{z}, dV=s² sin θdθdϕds.

New parameters (l, m, n) are defined as follows:

$$l = sin θ cos ϕ \\ m = sin θ sin ϕ \\ n = cos θ = \sqrt{1 - {l}^{2} - {m}^{2}} \qquad ( 11 )$$

dV can be derived from the following equation.

$$dV = {s}^{2} dldmds / n \qquad ( 12 )$$

Eq. (12) is substituted into Eq. (10) to attain the following equation.

$$\vec{V} ( \vec{{D}_{ab}} ) = {\int \int \int }_{V} I ( \vec{s} ) \frac{{e}^{- j 2 π \vec{{D}_{ab}} \cdot \underline{\hat{s}}}}{n} dldmds \qquad ( 13 )$$

The component vector \vec{D_{ab}} of a baseline vector in a Cartesian coordinate system is (\vec{u_{ab}},\vec{v_{ab}}):

$$\vec{{u}_{ab}} = ( \vec{{x}_{b}} - \vec{{x}_{a}} ) / {λ}_{o} \\ \vec{{v}_{ab}} = ( \vec{{y}_{b}} - \vec{{y}_{a}} ) / {λ}_{o} \qquad ( 14 )$$

Because the terahertz antennas (the receiving positions of the receiving antennas) are arranged at the same height, the visibility scattering function of the organ (breast) becomes the following equation.

$$\vec{V} ( {u}_{ab} , {v}_{ab} ) = {\int }_{l} {\int }_{m} {\int }_{n} \frac{I ( s , l , m )}{\sqrt{1 - {l}^{2} - {m}^{2}}} {e}^{- j 2 π ( {u}_{ab} l + {v}_{ab} m )} dldmds \qquad ( 15 )$$

The line integral along a radial coordinate n is as follows.

$${\tilde{I}}^{( p , q )} ( l , m ) = {\int }_{s} \frac{{I}^{( p , q )} ( s , l , m )}{\sqrt{1 - {l}^{2} - {m}^{2}}} ds \qquad ( 16 )$$

Through Eq. (16), a visibility scattering function of the following two-dimensional integral for a variable (l, m) is obtained.

$$\vec{V} ( {u}_{ab} , {v}_{ab} ) = \int \int \tilde{I} ( l , m ) {e}^{- j 2 π ( {u}_{ab} l + {v}_{ab} m )} dldm \qquad ( 17 )$$

The visibility scattering function (17) is a two-dimensional Fourier transform, so it is possible to reconstruct the 2D image of the breast through an inverse Fourier transform.

$$I = \int \int \vec{V} ( {u}_{ab} , {v}_{ab} ) {e}^{j 2 π ( {u}_{ab} l + {v}_{ab} m )} dudv \qquad ( 18 )$$

Eq. (18) shows that it is possible to reconstruct a 2D image of a 3D organization model by way of performing an inverse Fourier transform on the visibility scattering function.

In the above step S3, in the case that the organ is non-magnetic and conductive, the scattered electric field received by the organ at the receiving position of any receiving antenna can be calculated through Eq. (6), so the method can be used to monitor various physiological and pathological characteristics of tissues, such as breast tumors, cerebral strokes and skin diseases.

In the above step S4, it is achievable to form a time series of at least one electromagnetic properties of the organ, based on the scattered electric field detected at two of the receiving antenna positions in at least three receiving positions, and calculate out a difference between the electromagnetic properties detected by at least two receiving antenna positions, so as to construct a 2D image of the organ. It is achievable to keep the heights of at least three receiving positions as same as the height of the organ (that is, the three receiving positions have the same distance in the vertical direction as the vertical height of the organ), meanwhile gradually change the height (that is, moving the receiving antenna in the vertical direction to change the receiving position), and calculate out a difference between the electromagnetic properties detected by at least two receiving positions (electromagnetic property distribution information, specifically amplitude and phase position information of electromagnetic property distribution), so as to construct a 3D image of the target organ. It is achievable to form a complete set of data by way of calculating the distribution of the visible intensity difference of the organ obtained by the receiving antenna at different heights, and comparing the visible intensity difference obtained by the receiving antenna at the different heights, so as to realize 3D image reconstruction. The spatial resolution of the 3D image is influenced by antenna types, antenna shapes, scanning speed, scanning height, operating frequency, and antenna materials.

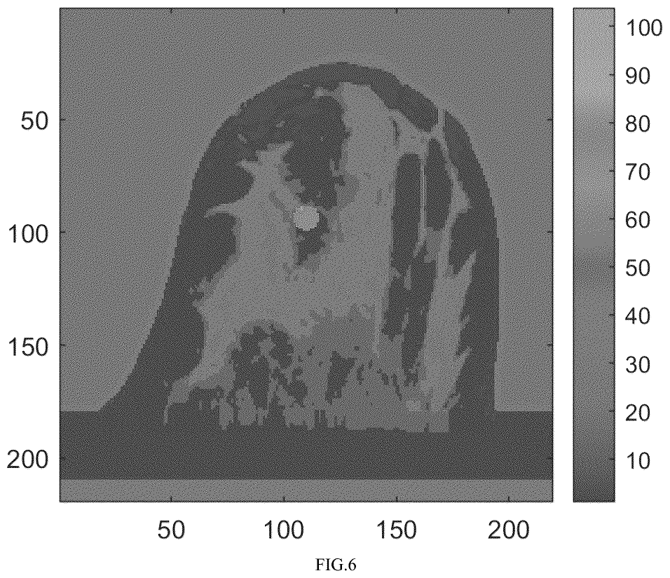

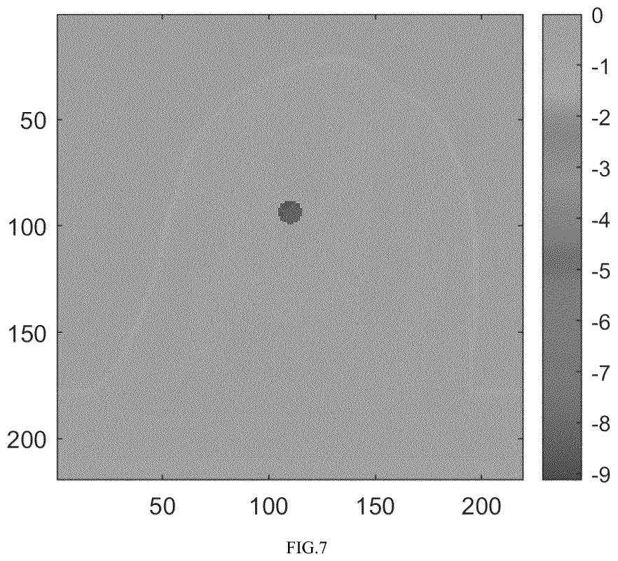

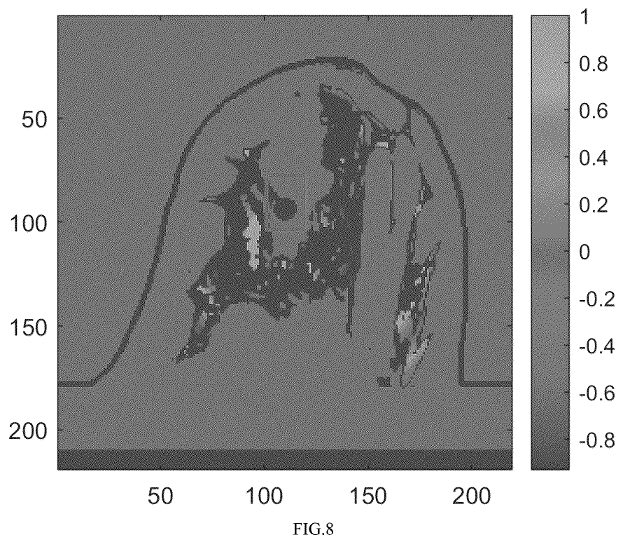

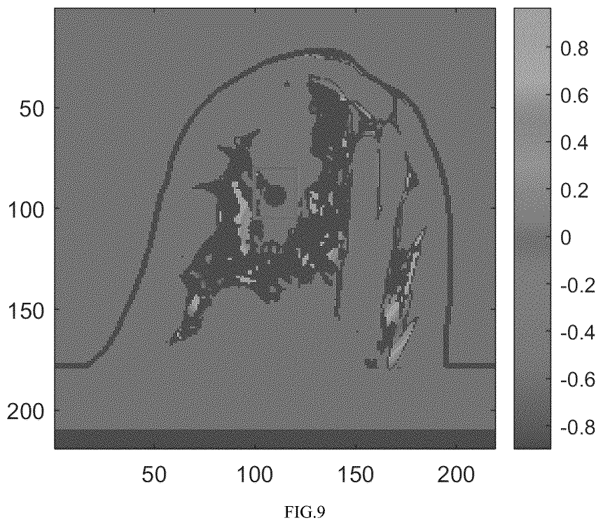

In order to verify the holographic terahertz imaging method proposed by the present invention, a 3D simulation model is established through a MATLAB platform for simulating an influence of different tissues on scattering electric fields at occurrence of breast tumors. FIG. 6 is a 2D image (real part) of the 3D breast model in the example of the present invention. FIG. 7 is a 2D image (imaginary part) of the 3D breast model in the example of the present invention. FIG. 8 is a 2D reconstructed image (real part) of the 3D breast model in the example of the present invention. FIG. 9 is a 2D reconstructed image (imaginary part) of the 3D breast model in the example of the present invention. The reconstructed images of the 3D breast model clearly show diverse breast tissues, including tumor cells.

The above content is only some preferred examples of the present invention and is not intended to limit the present invention, which may have various alterations and variations for a person skilled in the art. Any modifications, equivalent replacements, improvements and the likes made within the spirit and principles of the present invention shall fall within the protection scope of the present invention.

What is claimed is:

1. An ultra-wideband terahertz imaging system comprising:
a terahertz wave generator used to generate a continuous ultra-wideband terahertz signal;
a terahertz signal emitter emitting the continuous ultra-wideband terahertz signal generated by said terahertz wave generator to an organ;
a terahertz biological signal receiver having a receiving antenna adjustable at a vertical height, and used to receive an electromagnetic field around the organ to obtain a scattered electric field;
a terahertz biological signal processor performing multi-dimensional image reconstruction on the received scattered field electric field to obtain a multi-dimensional image of the organ;
an image displayer used to display the multi-dimensional image of the organ; and
an intelligent scanning controller connected with and controlling said terahertz wave generator, said terahertz signal emitter, said terahertz biological signal receiver, said image displayer, and said terahertz biological signal processor.

2. The ultra-wideband terahertz imaging system according to claim 1, wherein said terahertz signal emitter comprises at least one emitting antennas, and said terahertz biological signal receiver comprises at least one receiving antennas.

3. The ultra-wideband terahertz imaging system according to claim 1, wherein said intelligent scanning controller controls the receiving antenna of said terahertz biological signal receiver to move around the organ, so as to perform rotational scanning, or controls the receiving antenna of said terahertz biological signal receiver to move upwards and downwards, so as to perform scanning in a vertical direction, or controls the receiving antenna of said terahertz biological signal receiver to move horizontally, so as to perform scanning in a horizontal direction.

4. An imaging method of the ultra-wideband terahertz imaging system according to claim 1, comprising the steps of
S1, controlling said terahertz wave generator to generate a continuous ultra-wideband terahertz signal;
S2, controlling said terahertz signal emitter to uninterruptedly emit an ultra-wideband terahertz signal to the organ;
S3, controlling said terahertz biological signal receiver to receive an electromagnetic field from the organ, so as to obtain a scattered electric field;
S4, controlling said terahertz biological signal processor to perform multi-dimensional image reconstruction on the scattered electric field detected by said terahertz biological signal receiver, so as to obtain a multi-dimensional image of the organ; and
S5, transmitting the multidimensional image of the organ to said image displayer for imaging the tested organ.

5. The imaging method according to claim 4, wherein S2 comprises the sub-steps of
S21, establishing a rectangular coordinate system at the area to be imaged where the organ is situated; and
S22, enabling N_{T} emitting antennas of said terahertz signal emitter that surround the organ or are situated at one or both sides of the organ, to uninterruptedly apply an ultra-wideband terahertz wave signal to the organ, and setting N_{T} as ≥1.

6. The imaging method according to claim 5, wherein when the number of said emitting antennas, N_{T}>1 occurs, and said emitting antennas are distributed in a uniform circular shape, each transmitting antenna makes an excitation to generate an incident electric field in proper order, and an entire incident electric field is a sum of the incident electric fields excited by N_{T} emitting antennas.

7. The imaging method according to claim 4, wherein during processing a 2D image, S3 comprises the sub-steps of
S31, controlling one receiving antenna of said terahertz biological signal receiver to detect a scattered electric field of at least three receiving positions at the same vertical height of the receiving antenna away from the organ; or controlling at least three receiving antennas at the same vertical height at a distance from the organ to detect a scattered electric field of other receiving positions;
S32, moving the organ out of the detected area to obtain an incident electric field of said emitting antenna; and
S33, based on the incident electric field of said emitting antenna and the scattered electric field detected by said receiving antenna, calculating out a scattered electric field echo.

8. The imaging method according to claim 4, wherein during processing a 3D image, S3 comprises the sub-steps of
S31, controlling one receiving antenna of said terahertz biological signal receiver to detect a scattered electric field of at least three receiving positions at the same vertical height of the receiving antenna away from the organ; or controlling at least three receiving antennas at the same vertical height at a distance from the organ to detect a scattered electric field of other receiving positions;
S32, moving the organ out of the detected area to obtain an incident electric field of said emitting antenna;
S33, based on the incident electric field of said emitting antenna and the scattered electric field detected by said receiving antenna, calculating out a scattered electric field echo; and
S34, adjusting a vertical height of said receiving antenna, then re-executing the sub-steps S31 to S33.

9. The imaging method according to claim 4, wherein during processing a 2D image, S4 comprises the sub-steps of
S41, establishing a nonlinear mathematical model involving electromagnetic properties such as dielectric constants, conductivity and magnetic permeability of the organ and the scattered electric field echo, based on the distribution of the receiving positions of said receiving antennas, establishing a mathematical model representing an internal structure of the organ;
S42, sequentially comparing the scattered electric field echoes obtained at any two receiving positions among all receiving positions of said receiving antennas situated at the same vertical height, and obtaining information reflecting an amplitude and phase position of the electromagnetic property distribution of the organ; and
S43, according to the continuously-detected electromagnetic property distribution information, extracting a corresponding varied value and curve from the established nonlinear mathematical model and the mathematical model representing the internal structure of the organ, and based on the varied values, reconstructing a 2D image of the organ.

10. The imaging method according to claim 4, wherein during processing a 3D image, S4 comprises the sub-steps of
S41, establishing a nonlinear mathematical model involving electromagnetic properties such as dielectric constants, conductivity and magnetic permeability of the organ and the scattered electric field echoes, and based on the distribution of the receiving positions of said receiving antennas, establishing a mathematical model representing an internal structure of the organ;
S42, sequentially comparing the scattered electric field echoes obtained at any two receiving positions among all receiving positions of said receiving antennas situated at the same vertical height, and obtaining information reflecting an amplitude and phase position of the electromagnetic property distribution of the organ;
S43, sequentially comparing the scattered electric field echoes obtained at any two receiving positions among all receiving positions of said receiving antennas adjusted in height, and situated at the same vertical height, and obtaining information reflecting an amplitude and phase position of the electromagnetic property distribution of the organ; and
S44, according to the continuously-detected electromagnetic property distribution information, extracting a corresponding varied value and curve from the established nonlinear mathematical model and the mathematical model representing the internal structure of the organ, and based on the varied values, reconstructing a 3D image of the organ.
